(19) United States
(12) Patent Application Publication
(10) Pub. No.: US 2020/0261262 A1
(43) Pub. Date: Aug. 20, 2020

(54) A COOLING SYSTEM FOR COOLING AN EXTREMITY OR BODY PART

(71) Applicant: BrainCool AB, Lund (SE)

(72) Inventors: Martin WALEIJ, Lund (SE); Christian STRAND, Staffanstorp (SE); Ola STRÖMBERG, Dalby (SE); Jonas BLANKING, Malmö (SE)

(73) Assignee: BrainCool AB, Lund (SE)

(21) Appl. No.: 16/760,826

(22) PCT Filed: Oct. 31, 2018
(86) PCT No.: PCT/EP2018/079889

§ 371 Date: Apr. 30, 2020

(30) Foreign Application Priority Data
Nov. 1, 2017 (SE) 1751358-1

(51) Int. Cl. A61F 7/02; A61F 7/10

(52) U.S. Cl. CPC A61F 7/02; A61F 2007/0046; A61F 7/10

(57) ABSTRACT

A cooling system (10) for cooling at least one extremity, such as a finger or toe, or body part of a human is provided. The cooling system (10) comprises at least one malleable thermal conductive viscoelastic material (11), adapted to be formed into a homogenous body around and fully covering the at least one extremity or body part, thereby providing for a complete and continuous direct contact between the malleable thermal conductive viscoelastic material and the at least one extremity or body part. Moreover, the cooling system (10) comprises a cooling circuit (12) for providing cooling at least partly around said homogenous body of malleable thermal conductive viscoelastic material (11).

### TECHNICAL FIELD

The present invention relates to a cooling system for cooling at least one extremity, such as the finger(s) or toe(s), or body part of a patient. More particularly, the cooling system is provided to alleviate or reduce peripheral neuropathy for patients e.g. undergoing chemotherapy or radiation therapy.

### BACKGROUND

Peripheral neuropathy relates to the damage to or disease affecting nerves, which may impair sensation, movement, gland or organ function, or other aspects of health, depending on the type of nerve affected.

Peripheral neuropathy may arise from chemotherapy or radiation therapy, and it is particularly noticed in the extremities of the body, e.g. the hand or feet, but also other extremities such as the head region including the scalp, and neck may be affected. Peripheral neuropathy may cause severe painful cramps, fasciculations (fine muscle twitching), muscle loss, bone degeneration, and changes in the skin, hair, and nails. In some cases the nerve damages due to peripheral neuropathy may be permanent. Although a lot a research is put into the treatment of or reduction of peripheral neuropathy, there is a need to provide solutions to reduce, alleviate or prevent the associated nerve damage.

### SUMMARY

An object of the invention is to overcome or at least alleviate the problems associated with prior art.

According to a first aspect a cooling system for cooling at least one extremity, such as a finger or toe, or body part of a human is provided. The cooling system comprises at least one malleable thermal conductive viscoelastic material, adapted to be formed into a homogenous body around and fully covering the at least one extremity or body part, thereby providing for a complete and continuous direct contact between the malleable thermal conductive viscoelastic material and the at least one extremity or body part. Moreover, the cooling system comprises a cooling circuit for providing cooling at least partly around said homogenous body of malleable thermal conductive viscoelastic material.

### BRIEF DESCRIPTION OF THE DRAWINGS

By way of example, embodiments of the present invention will now be described with reference to the accompanying drawings, in which:

FIG. 1 is a schematic drawing of a cooling system for cooling at least one extremity of a person;

FIGS. 2a to 2j shows different stages of providing the malleable thermal conductive viscoelastic material around the extremity in use;

FIG. 3 shows the cooling system with a part of a cooling circuit provided around the homogenous body of the malleable thermal conductive viscoelastic material;

FIGS. 4a to 4j shows different stages of assembling the cooling system in use, wherein a first spacer is provided between the fingers of a patient;

FIG. 5 shows a cross section of the assembled cooling system wherein the cooling circuit comprises a cooling pad providing cooling utilizing a cooling fluid flowing on either side of the extremities in use;

FIG. 6 shows a cross section of the assembled cooling system wherein the cooling circuit comprises a cooling pad providing cooling utilizing a cooling fluid flowing in a separate flow paths around the extremities in use;

FIG. 7 shows a cross section of the assembled cooling system wherein the cooling circuit provides for direct cooling utilizing a cooling medium flowing in direct contact with the outer surface of the homogenous body of malleable thermal conductive viscoelastic material;

FIG. 8 shows a cross section of the assembled cooling system provided with a first space wherein the cooling circuit comprises a cooling pad providing cooling utilizing a cooling fluid flowing on either side of the extremities in use;

FIG. 9 shows a cross section of the assembled cooling system provided with a first spacer wherein the cooling circuit comprises a cooling pad providing cooling utilizing a cooling fluid flowing in a separate flow paths around the extremities in use;

FIG. 10 shows a cross section of the assembled cooling system provided with a first spacer wherein the cooling circuit provides for direct cooling utilizing a cooling medium flowing in direct contact with the outer surface of the homogenous body of malleable thermal conductive viscoelastic material;

FIGS. 11a and 11b respectively shows a cooling system when provided with a cover;

FIGS. 12a and 12b respectively shows a cooling system when provided with a cover; and

FIGS. 13a to 13c respectively shows a malleable thermal conductive viscoelastic material having an outer coating, when provided to the extremity at different stages.

### DETAILED DESCRIPTION

The present invention will now be described in more detail.

The present inventors have realized that by cooling the extremities or body parts, e.g. during chemotherapy or radiation therapy, may reduce the condition.

Medical cooling is used in many other medical applications.

One key aspect of the present invention is to provide a cooling system allowing for an optimal direct contact surface between a cooling medium and the body part, e.g. extremity to be treated. By providing an optimal contact surface, the cooling medium is capable of cooling the entire surface of the extremity or body part, without any gaps, air pockets or insulating layers in between. Insulating layers, e.g. a one-sized glove provided in direct contact with the hand, is not able to make a perfect contact to the fingers of the hand, due to the normal variations of patients hand autonomy, whereby insulating pockets or folds are formed between the glove and hand in use. These pockets or folds have a negative effect of the cooling efficiency, since the areas of the hand in communication with the pockets or folds will not be controllably cooled. Another aspect to consider is that, for various reasons, it is not desired to cool the extremity or body part using direct contact with e.g. a circulating fluid, since the skin of the extremity may be negatively affected during prolonged direct cooling with a cooling fluid. For example the skin may loosen causing increased nerve pain to the patient.

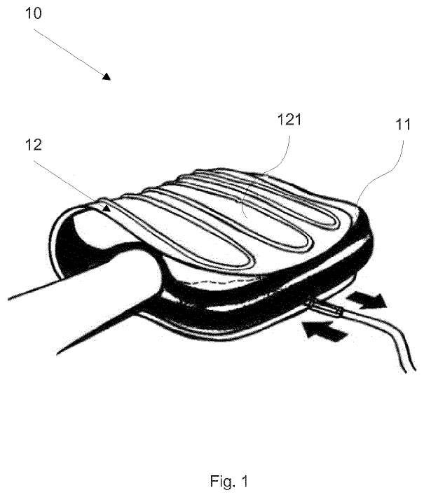

FIG. 1 shows a cooling system 10 for cooling at least one extremity, such as a finger or toe, of a human. The cooling system 10 comprises at least one malleable thermal conductive viscoelastic material 11, adapted to be formed into a homogenous body around and fully covering the at least one extremity the at least one extremity. The homogenous body provides for a complete and continuous direct contact between the malleable thermal conductive viscoelastic material and the at least one extremity. The cooling system 10 further comprises a cooling circuit 12 for providing cooling at least partly around said homogenous body of malleable thermal conductive viscoelastic material 11.

A key characteristic of the malleable thermal conductive viscoelastic material is that it is malleable, i.e. capable of being altered or controlled by outside forces or influences, and having a capacity for adaptive change.

The malleable thermal conductive viscoelastic material behaves like a fluid dough, being plastically deformable and joinable around the extremity in use. It may be made of a material which allows for bonding to the extremity. The bonding force to the extremity may be less than the intrinsic bonding force between the molecules of the material. In this way the malleable thermal conductive viscoelastic material may be easily be peeled off the extremity after use, while allowing for providing a liquid like continuous contact surface to the extremity when formed into a continuous body around the extremity in use.

Moreover, another key characteristic of the malleable thermal conductive material is that it comprises a composition able to be adapted or formed into a homogenous shape, thereby having a dough consistency, such that molecules of the composition may bind to other molecules of the composition, without cracks, gaps or dividing surfaces forming therebetween. This dough consistency allows the malleable thermal conductive material to act as a flexible attachment medium with the capability of filling out voids, gaps, or spaces between itself and the extremity. In other words the malleable thermal conductive material acts as a void, gap, or space remover, adapted to filling out each surface, including pore(s) of the extremity to be cooled.

The direct contact between the malleable thermal conductive viscoelastic material is achieved without any other material composition, such as a coating, present between the extremity and the malleable thermal conductive viscoelastic material.

The malleable thermal conductive viscoelastic material may be a synthetic slime. As such the malleable thermal conductive viscoelastic material may comprise a composition containing polyvinyl alcohol, and boric acid, borax or borate buffer as key ingredients of the synthetic slime. Additionally, the malleable thermal conductive viscoelastic material further may further comprise a thickening agent, e.g. that comprised in a conventional shaving gel, for thickening and providing form stability purposes. Optionally, the malleable thermal conductive viscoelastic material may comprise a plasticizer, e.g. comprised in a conventional lotion for providing improved stretchability.

Alternatively, the thermal conductive viscoelastic material may be a dough, gel, hydrogel, wax, or foam.

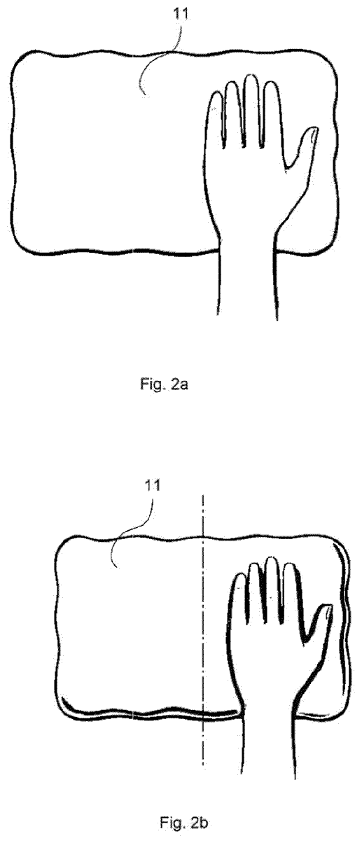

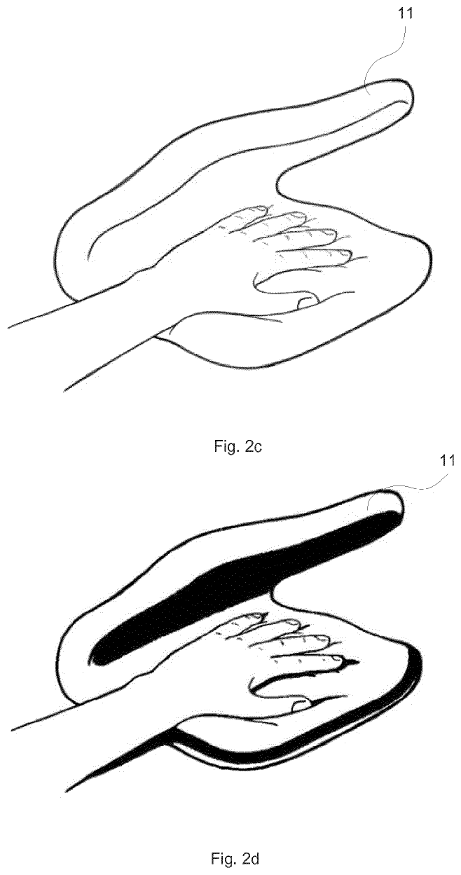

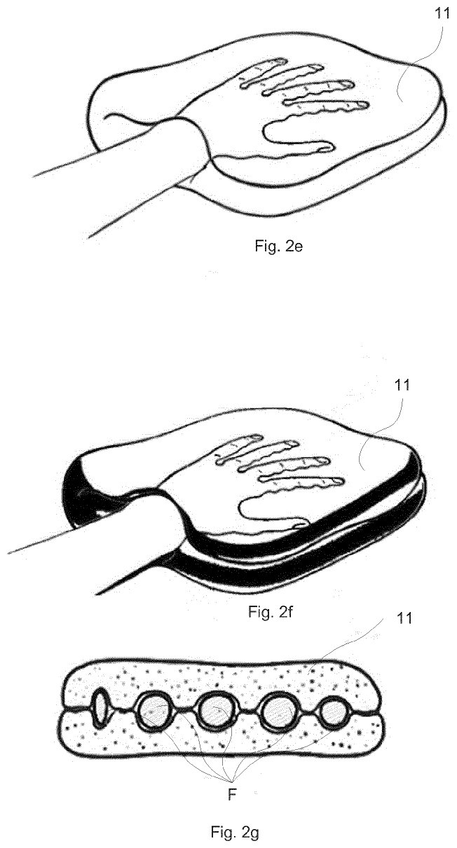

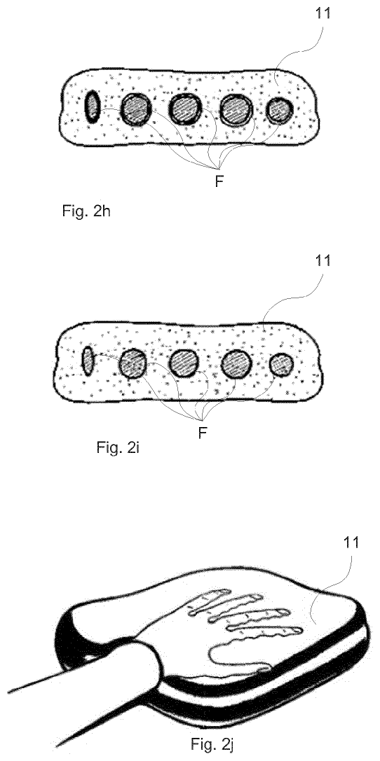

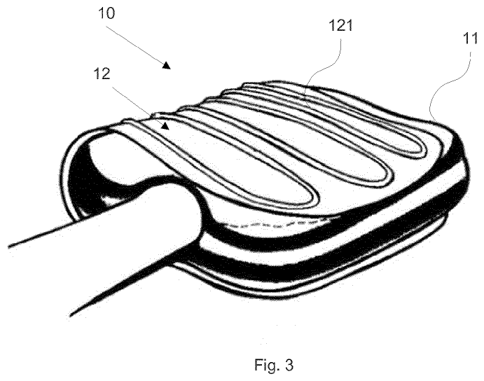

With reference to FIGS. 2a to 2g the malleable thermal conductive viscoelastic material may be provided as a sheet comprising an upper surface and a lower surface prior to forming the sheet into the homogenous body fully covering the at least one extremity.

For example, this sheet may be provided similar to the dough of a known pizza kit, where the sheet having a uniform thickness is rolled up into a roll e.g. using a separating material, e.g. parchment paper. This saves space during transport.

At the start of the cooling process, this sheet may be folded directly around the extremity as shown with reference to FIGS. 2a to 2b, without any intermediate material provided therebetween.

The sheet is then formed, e.g. by applying a force around each extremity, e.g. finger, by kneading or massaging the sheet around each extremity.

FIGS. 2c and 2d respectively shows the sheet during folding whereas FIG. 2e to FIG. 2g respectively shows the sheet just after folding. FIG. 2h shows the sheet after folding but before final external forces are applied to remove any gaps between the extremities. FIGS. 2i to 2j shows the malleable thermal conductive material after forming, i.e. applying an external pressure or force, whereby said material is formed into a homogenous body provided in direct contact with the extremities, such that no gaps or cracks are provided therebetween. Hence, as may be noted from FIGS. 2i and 2j, after application of the external force the folded sheet forms a homogenous body completely surrounding each extremity.

The malleable thermal conductive viscoelastic material may have a sticky texture to be able to attach to the extremity.

The malleable thermal conductive viscoelastic material 11 may also be mounted on a supporting membrane (not shown). The malleable material 11 is then attached to the at least one extremity or body part, as described above. Preferably, a force is applied by kneading or massaging the malleable material 11 mounted on the supporting membrane to shape it around the extremity. Direct contact between the malleable thermal conductive viscoelastic material 11 and the extremity of the patient is achieved due to the use of the malleable material, as described above.

An advantage using the malleable material mounted on the supporting membrane is that it may be applied and removed from the extremity several times. This is particularly advantageous for example when the patient has to visit lavatory facilities. Further, the supporting membrane may comprise channels for circulation of a cooling liquid therethrough (not shown).

The supporting membrane may be in the form of a bag (not shown). Typically, the bag consists of two superimposed sheets of flexible, fluid tight material, such as a plastic. The sheets are sealed together along their edges to form the bag. Sealing in a predetermined pattern throughout the area within the edges of the sheets may also occur. This forms channels for circulation of a cooling liquid therethrough (not shown).

With reference to the bag described above, the two superimposed sheets of e.g. plastic film may be sealed by means of welding (not shown).

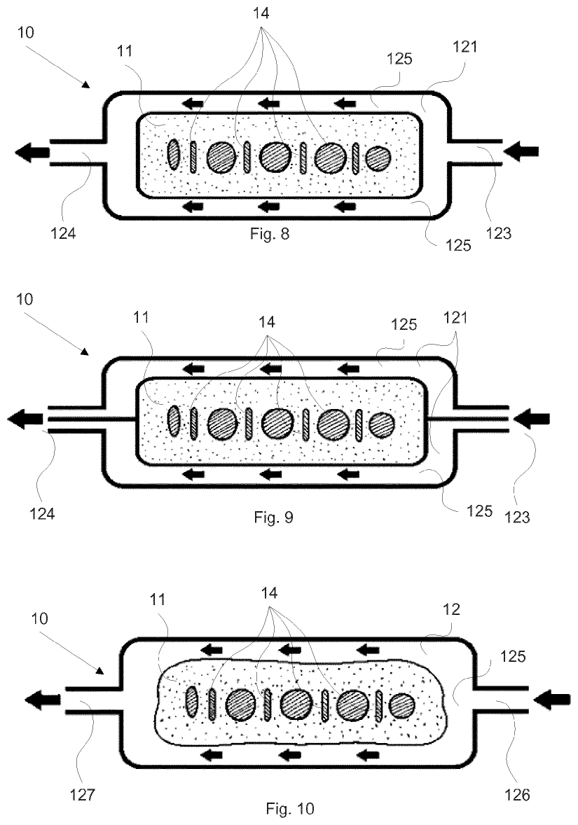

As shown with reference to FIGS. 1, 3, 4i, 4j, 5, 6, 8, and 9 the cooling circuit 12 may comprise a known flexible cooling pad 121 comprising a channel for circulation of a cooling fluid therethrough. Cooling pads are conventionally used in medical cooling processes for medically cooling various regions of the body of a person or patient in a non-invasive manner by placing the cooling pad onto or around the body part to be cooled. A cooling pad typically consists of two superimposed sheets of flexible, fluid tight material that are sealed together along their edges.

The cooling pad may contain a self contained cooling medium or fluid as that shown in FIGS. 3, 11a and 11b. Hence such a cooling pad may be applied to the homogenous body in a pre-cooled state.

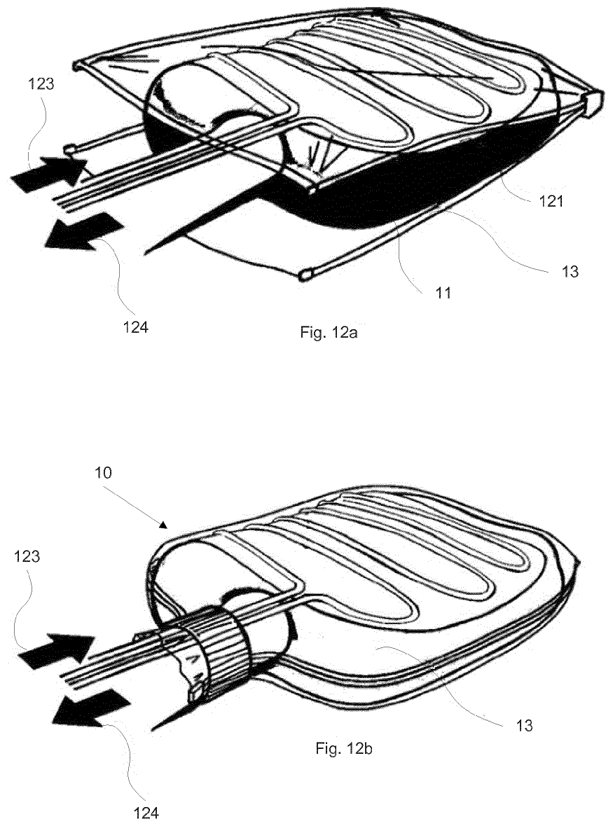

Optionally, the cooling pad may comprise an inlet port is arranged in one end of the cooling pad for letting a flow of cooling fluid, e.g. liquid, into the cooling pad and an outlet port is provided in another end allowing the flow of cooling liquid out of the pad, as shown with reference to FIGS. 1, 4i, 4j, 5, 6, 8, 9, 12a and 12b.

A flexible cooling pad is particularly preferred since in the same way as the malleable thermo conductive viscoelastic material is able to form a complete direct and continuous contact surface to the extremity, it is also able to form a complete direct and continuos contact surface to the cooling pad. Thereby the malleable thermo conductive viscoelastic material provides for a superior direct and continuous contact surface to the cooling pad, than would be possible if the cooling pad would be placed directly around the extremity without the malleable thermo conductive viscoelastic material provided therebetween.

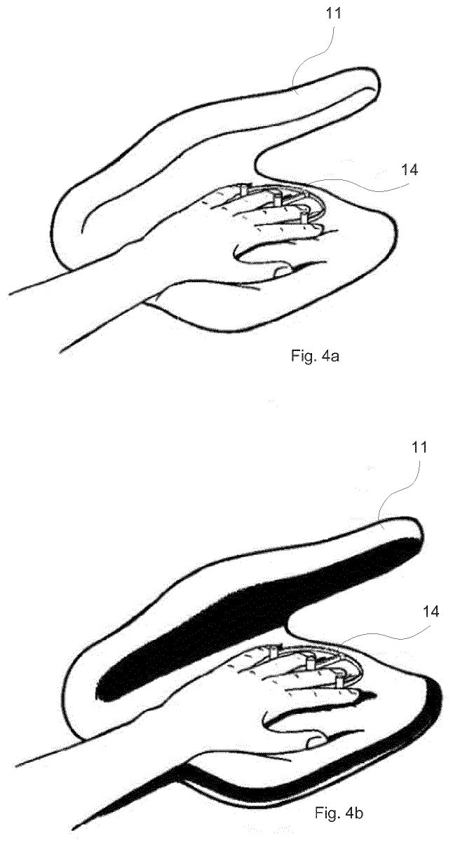

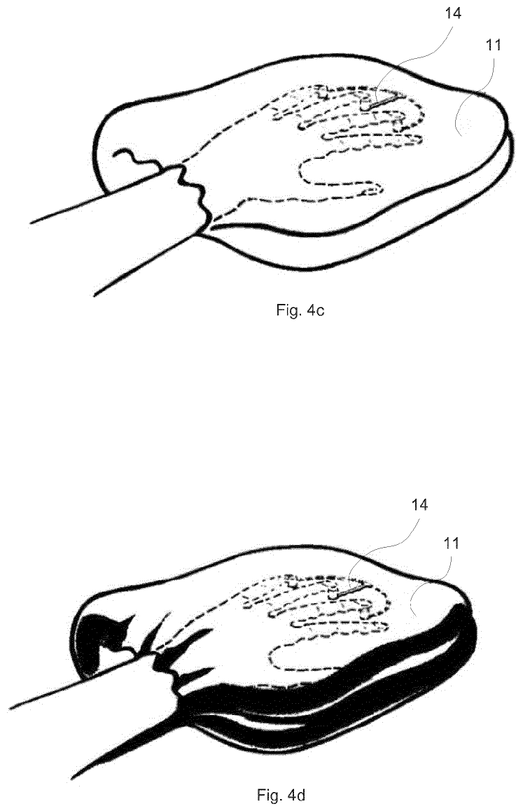

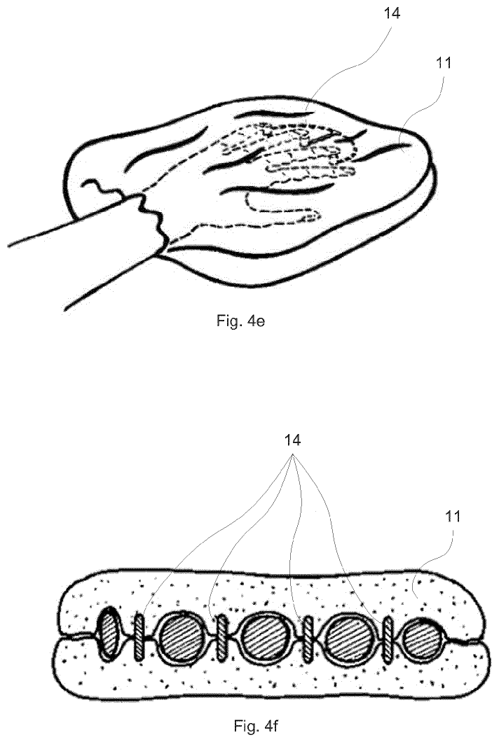

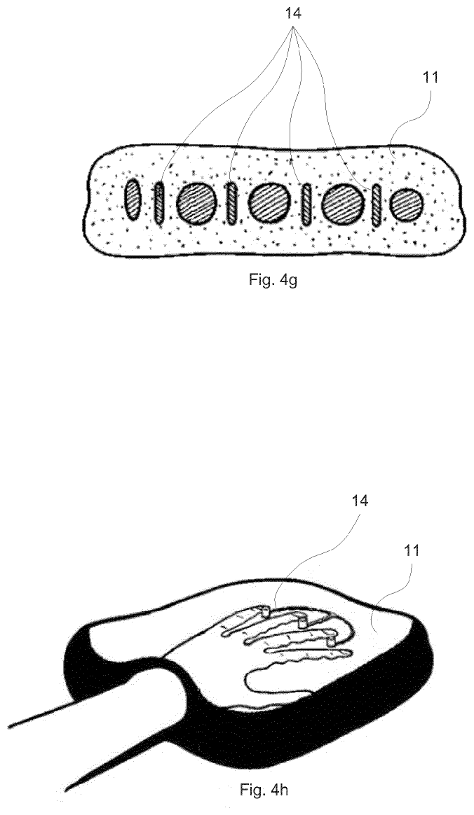

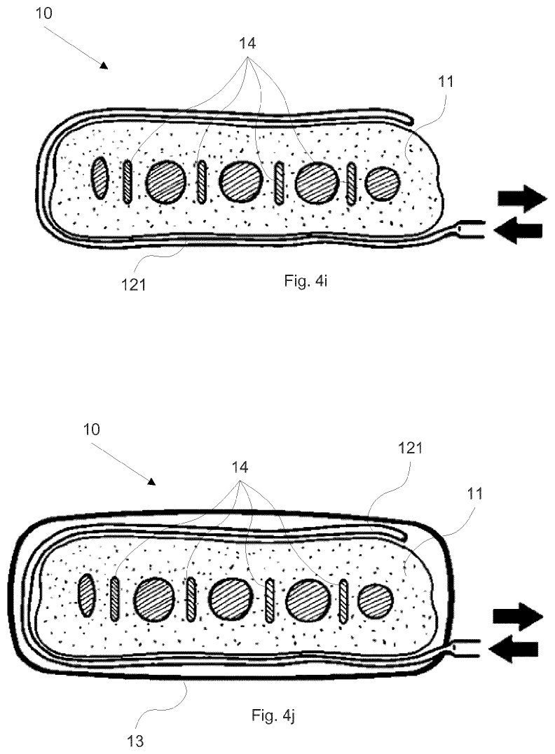

As shown with reference to FIGS. 4j, 11a, 11b, 12 and 12b the cooling system 10 may further comprise an outer cover 13 for housing the malleable thermal conductive viscoelastic material 11 and at least part of the cooling circuit 12 provided around the homogenous body thereof.

As mentioned above, with reference to the cooling pad 121, the cooling circuit 12 comprises an inlet 123 for introducing a cooling liquid into a space provided between the cover and the malleable thermal conductive viscoelastic material and an outlet 124 allowing cooling liquid introduced into the space to exit said space.

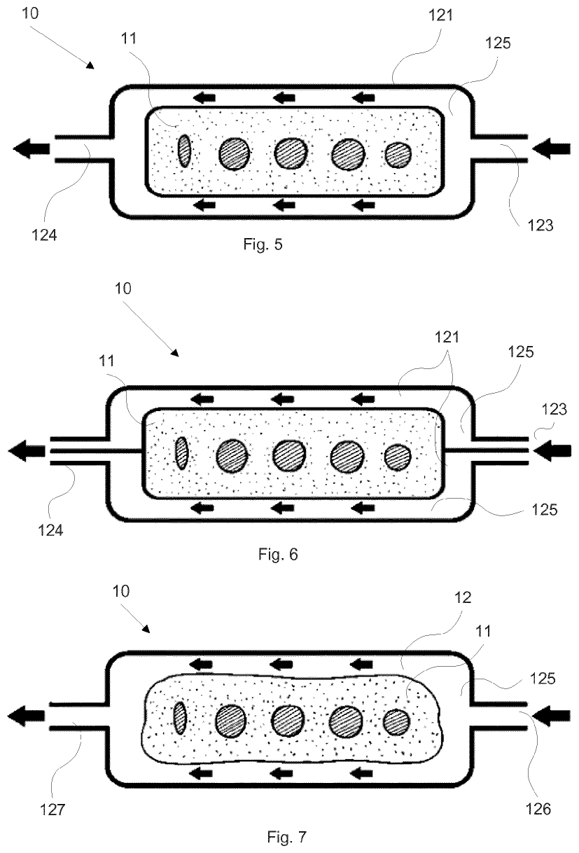

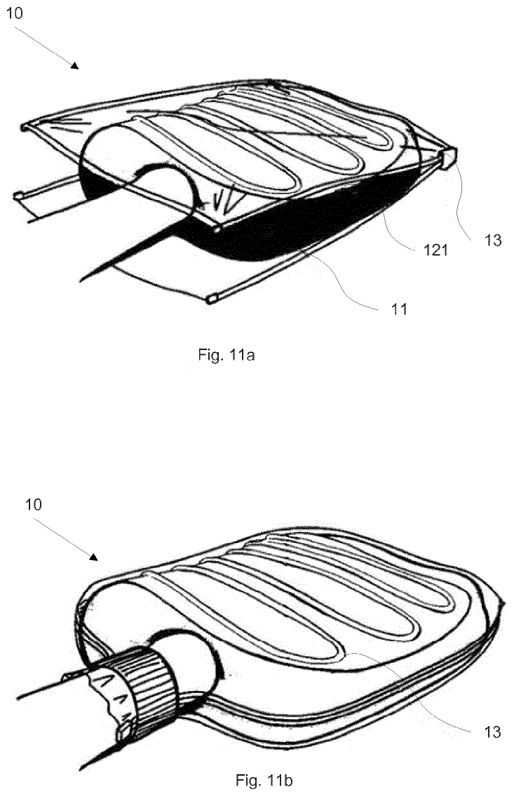

The space 125 could be a space 125 internal to the cooling pad, as shown with reference to FIGS. 5, 6, 8, and 9 but in a case where no cooling pad is used, such as that shown with reference to FIGS. 7 and 10, the space 125 is formed between the outer periphery of the homogenous body of the malleable thermal conductive viscoelastic material.

With reference to FIGS. 4a to 4j a first spacer 14 may be provided between at least two extremities. In this way the first spacer 14 is arranged at a distance, larger than zero, from each extremity, where the malleable thermal conductive viscoelastic material fully covering each extremity fills out this distance between the first spacer and each extremity. In this way the homogenous body of the malleable thermal conductive viscoelastic material remains homogenous between the at least two extremities in use.

It thus be noted that the first spacer 14 is not provided in direct contact with the extremity. The purpose of the first spacer 14 is to allow the patient to move his/her extremities during the cooling process to a certain extent, while making sure that the extremities of the patient is not moved to such an extent that the extremities would come into direct contact or in too close vicinity of each other, since that would have a negative effect on the cooling process. One should bear in mind that in this context the extremities, such a fingers or toes of the patient, act as a source of heat, and the closer they are provided together the better they resist the cooling.

The first spacer 14 may advantageously be made in of a thermal conductive material, such as but not including metallic or plastic material.

In FIGS. 4a to 4j the first spacer 14 is provided as a separate entity, without any inherent means of reducing its own temperature. However, it should be noted that the first spacer 14 could be connected to heat pump transferring heat from the area surrounding the first spacer 14 to the outside of the cover. Hence in such an example, the first spacer 14 may internally comprises a series of coils, e.g. forming a single fluid transferring channel, just like the inside of a refrigerator. An inlet may be connected to one end of the channel and an outlet may be connected at the other end of the channel. The inlet and outlet can be connected using a tubing connected to the heat pump provided outside the cover. As it makes a circuit through them, it changes back and forth from a liquid to a gas.

Although not shown, a second spacer may be provided between the outer perimeter of the homogenous body and the cover 13 to provide a space 125 for accommodating at least part of the cooling circuit 12.

Similarly, to the first spacer 14 the second spacer may be provided in a thermal conductive material. It could also be connected to a heat pump for providing cooling around the area in vicinity of the second spacer.

It should be appreciated that as an alternative to providing the cooling circuit with a cooling pad, it is also possible to distribute a cooling fluid directly around the homogenous body of the malleable thermal conductive viscoelastic material 11. Such a solution is shown with reference to FIGS. 7 and 10. In such an example, instead of a cooling pad, the cooling circuit 12 may comprise an inlet 126 provided through cover 13 for introducing a cooling fluid into the space 125 formed, e.g. by means of the second spacer 15, between the homogenous body of malleable thermal conductive viscoelastic material 11 and the cover 13, and an outlet 127 provided through the cover 13 for allowing cooling fluid to exit said space 125.

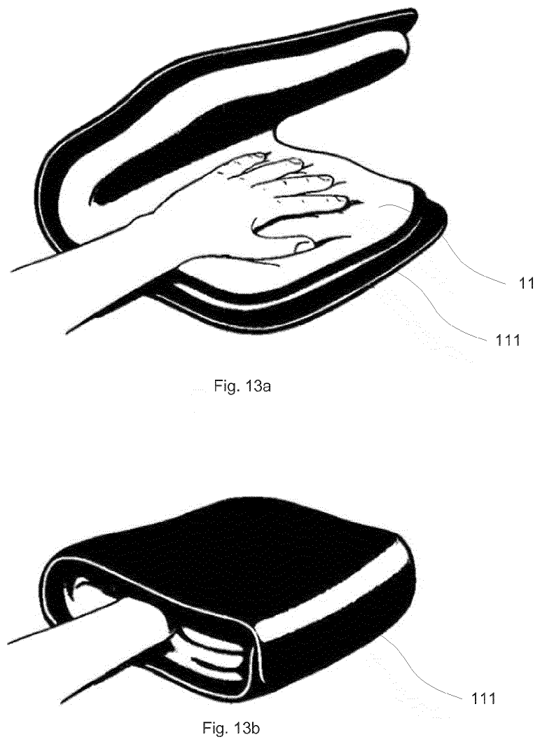

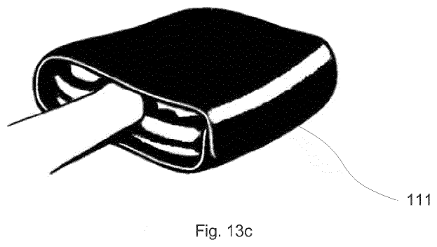

In such an example, in order to prevent the cooling medium to dissolve the outer boundary of the homogenous body, as shown with reference to FIGS. 13a and 13c, the one of the upper and lower surfaces of the sheet of thermal conductive viscoelastic material 11 is provided with a coating 111 made of a material being unsolvable to the cooling fluid, whereby the side on which the coating is provided forms an outer coating of the homogenous body of malleable thermal conductive viscoelastic material in use, whereby the coating is provided on the side of the sheet facing away from the extremity in use. In use, the side of the sheet not provided with the coating 111 is facing towards the extremity while the other side provided with the coating 111 is facing away from the extremity. As shown in FIG. 13a, the coating 111 may protrude laterally on either side of the sheet of malleable thermal conductive material 11, such that when the sheet is folded around the extremity the respective ends of the coating 111 may be folded on top of each other thereby providing a seal for protecting the malleable thermal conductive viscoelastic material from the cooling medium flowing in direct contact with the coating 111 in the space 125, in use. Such a configuration is shown with reference to FIGS. 13b and 13c. In FIG. 13b the malleable thermal conductive material has not yet been subject to the final external forces forming it to a homogenous body. When the final external forces have been applied, e.g. by kneading the malleable thermal conductive viscoelastic material, the resulting homogenous body thereof is shown in FIG. 13c.

Depending on the cooling fluid used, the coating 111 may be unsoluble to water or alcohol. As an example, it may be made of silicon.

Alternatively, the malleable thermal conductive viscoelastic material may be formed by mixing a dry granulate of what is to be transformed into the malleable material with a plasticizer, such as water, before treatment. After a predetermined time, such as after 3 to 10 minutes, the dry granulate and the water has formed a gel being the malleable thermal conductive viscoelastic material which can be used as described above. This means that the malleable material may be delivered in a dry state as a dry granulate consisting of granules or powder (not shown). When using dry granules or powder instead of an already wetted malleable material, the shelf life is prolonged to several years.

The weight is also decreased when using the dry granulate, such as about 100 g per cooling system compared to about 400 g per cooling system when using an already wetted malleable material. A lower weight of the malleable material results in lower transporting costs. Typically, a number of cooling units used for each treatment is about 4 cooling systems 10.

Although some examples above have been made to extremities, such as the fingers of the hands, or toes of the feet, it should be appreciated that the cooling system using adopting the malleable thermal conductive viscoelastic material may be applied to any body part, such as a head or part of the head such as a scalp, or neck or any other body parts or regions of the human body. Further, the malleable thermal conductive viscoelastic material may be applied to only portions of the cooling system to ease direct contact between the cooling system and a specific area of the body part, such as the carotid artery of the neck.

1. A cooling system for cooling at least one extremity or body part of a human, comprising:
at least one malleable thermal conductive viscoelastic material, adapted to be formed into a homogenous body around and fully covering the at least one extremity or body part, thereby providing for a complete and continuous direct contact between the malleable thermal conductive viscoelastic material and the at least one extremity or body part; and
a cooling circuit for providing cooling at least partly around the homogenous body of malleable thermal conductive viscoelastic material.

2. The cooling system as defined in claim 1, wherein the malleable thermal conductive viscoelastic material is a paste, a wax, a gel, a foam, or a synthetic slime.

3. The cooling system as defined in claim 1, wherein the direct contact between the malleable thermal conductive viscoelastic material and the at least one extremity or body part is achieved without any other material composition present between the at least one extremity or body part and the malleable thermal conductive viscoelastic material.

4. The cooling system as defined in claim 1, wherein the malleable thermal conductive viscoelastic material is provided as a sheet comprising an upper surface and a lower surface prior to forming the sheet into the homogenous body fully covering the at least one extremity or body part.

5. The cooling system as defined in claim 1, wherein the malleable thermal conductive viscoelastic material provides a bonding force to the at least one extremity or body part which is less than the intrinsic bonding force between the molecules of the malleable thermal conductive viscoelastic material.

6. The cooling system as defined in claim 1, wherein the cooling circuit comprises a flexible cooling pad having a channel for circulation of a cooling fluid therethrough.

7. The cooling system as defined in claim 1, wherein the cooling circuit comprises an outer cover for housing the malleable thermal conductive viscoelastic material and at least part of the cooling circuit provided around the homogenous body.

8. The cooling system as defined in claim 7, wherein the cooling circuit comprises an inlet for introducing a cooling liquid into a space provided between the cover and the malleable thermal conductive viscoelastic material, and an outlet for allowing cooling liquid introduced into the space to exit from the space.

9. The cooling system as defined in claim 1, further comprising a first spacer provided between at least two extremities or body parts, such that the first spacer is arranged at a distance, larger than zero, from each extremity or body part by means of the malleable thermal conductive viscoelastic material fully covering each extremity or body part, whereby the homogenous body of the malleable thermal conductive viscoelastic material remains homogenous between the at least two extremities or body parts in use.

10. The cooling system as defined in claim 7, wherein a second spacer is provided between an outer perimeter of the homogenous body and the cover to provide a space for accommodating at least part of the cooling circuit.

11. The cooling system as defined in claim 1, wherein the malleable thermal conductive viscoelastic material comprises a composition containing polyvinyl alcohol, and boric acid, borax or borate buffer.

12. The cooling system as defined in claim 11, wherein the malleable thermal conductive viscoelastic material further comprises at least one of a thickening agent or a plasticizer.

13. The cooling system as defined in claim 4, wherein one of the upper and the lower surfaces, is provided with a coating made of a material being unsolvable to the cooling fluid, whereby the coating forms an outer coating of the homogenous body of malleable thermal conductive viscoelastic material in use, whereby the coating is provided on a side of the sheet facing away from the extremity or body part in use.

14. The cooling system as defined in claim 13, wherein the coating is unsoluable to water or alcohol.

15. The cooling system as defined in claim 14, wherein the coating is made of silicon.
